(19) United States
(12) Patent Application Publication
(10) Pub. No.: US 2014/0140683 A1
(43) Pub. Date: May 22, 2014

(54) Devices and Methods for Emanating Liquids

(71) Applicants: Helen Stephenson, Hull (GB); Richard Koontz, Hull (GB); Paul Newton, Hull (GB)

(72) Inventors: Helen Stephenson, Hull (GB); Richard Koontz, Hull (GB); Paul Newton, Hull (GB)

(73) Assignee: Reckitt & Colman (Overseas) Limited, Slough, Berkshire (GB)

(21) Appl. No.: 14/128,012

(22) PCT Filed: Jun. 25, 2012
(86) PCT No.: PCT/GB2012/051481

§ 371 Date: Jan. 31, 2014

(30) Foreign Application Priority Data
Jun. 24, 2011 (GB) 1110696.0

(51) Int. Cl. A61L 9/03; A01M 1/20

(52) U.S. Cl. CPC A61L 9/037; A01M 1/2077
USPC 392/395; 239/44

(57) ABSTRACT

A device for emanating a liquid is described wherein the device is for use with a refill of liquid, wherein the device comprises: a housing with at least one exit port for emanated liquid therein; a hollow and elongate extraction limb having at least one capillary tube therein, said at least one capillary tube being open at one end to extract liquid from the refill and open at the other end to deposit said liquid on at least one wick provided only on an outer surface of a refill housing.

### FIELD OF THE INVENTION

The present invention relates to devices and methods for improved airborne delivery of liquids containing one or more active materials wherein the active material comprises at least one of: a fragrance; an insecticide; a fungicide; a pesticide; a sanitising material; and/or a pharmaceutical.

### BACKGROUND

Liquids, and commonly volatile liquids, containing one or more active materials wherein the active material comprises at least one of: a fragrance; an insecticide; a fungicide; a pesticide; a sanitising material; and/or a pharmaceutical are delivered within the domestic environment via a variety of mechanisms. Devices are available with heaters disposed therein to increase the rate of emanation from a surface saturated with the liquid, such a surface could be a wick saturated with a fragranced liquid and the heater is located adjacent the wick surface and nearby a chimney to heat the liquid on the wick surface and cause it to more readily evaporate and disseminate into the surrounding environment through the chimney.

Common wick and heater emanation systems typically comprise a refill of liquid and an emanation device containing a heater. In these common systems the refill consists of a bottle of liquid, typically a volatile liquid, wherein the bottle is sealed with a wick holder that contains a central aperture which grips a wick that extends from the base of bottle and through the wick holder to extend a short distance above the holder; the part of the wick which extends above the holder is the exposed part of the wick from where all emanation of the liquid takes place. When the refill is connected to the emanation device, the wick extends into a chimney of the device and the device has a heater located adjacent to the chimney to heat the wick directly or indirectly via the chimney to cause evaporation of the liquid from the exposed part of the wick. In use evaporated liquid travels up the chimney and out into the surround environment, the liquid in the exposed part of the wick is replenished due to the wicking/capillary action of the wick drawing up more liquid from the bottle.

One drawback with known refills containing a wick is that the efficiency of a particular wick material depends on the nature of the liquid, or component liquids within the liquid. Most available wick materials, such as those mentioned above, have a limited porosity and liquid transfer rate/evaporation rate due, at least in part, to fractionation and/or blocking/clogging of the wick. Fractionation over time will change the character and/or intensity of the active and will slow evaporation, this is particularly noticeable for fragrances wherein common wicks cause the ‘high notes’ of a fragrance to be evaporated when the wick is first exposed to the fragranced liquid, and the ‘low notes’ are evaporated thereafter which affects the user's experience.

The present invention is concerned with providing an improved refill and emanation device for a wick and heater emanation system that addresses many of the drawbacks associated with such systems.

### SUMMARY OF INVENTION

According a first aspect of the present invention there is provided therefore a device for emanating a liquid wherein the device is for use with a refill of liquid, wherein the device comprises:

a housing with at least one exit port for emanated liquid therein;

a hollow and elongate extraction limb having at least one capillary tube therein, said at least one capillary tube being open at one end to extract liquid from the refill and open at the other end to deposit said liquid on at least one wick provided only on an outer surface of a refill housing.

Preferably the extraction limb has a single capillary tube therein. The extraction limb is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Whereas the capillary tube within the extraction limb is preferably provided with a diameter of between 1-2000 μm, and more preferably with a diameter of between 50-1500 μm, and even more preferably with a diameter of between 100-1000 μm, and most preferably with a diameter of 100 μm+/−50 μm.

The extraction limb is preferably provided with one or more vent holes. The extraction limb may be provided with one end in contact with the device and the end remote therefrom unfettered to, in use, enter the refill to provide venting and, to assist in ease of use, the end remote therefrom may be tapered to assist with entering the refill.

Alternatively, the extraction limb may be the capillary tube, in such an arrangement the exraction limb/capillary tube would be made out of a particularly strong material to resist damage by a user during operation.

The capillary tube(s) may be bent at its end adjacent to the device through an angle of between 45°-180° so that it may deposit, in use, the liquid from the refill reservoir on a wick provided on an outer surface of the refill housing. Preferably the capillary tube is bent through 180° to be located, in use, directly over a wick on the outer surface of the refill, advantageously this will ensure accurate delivery of liquid to the wick.

The capillary tube may be sized such that it has a length that can contact the liquid in the reservoir of a refill at one end and contact the at least one wick at the tube's other end. Alternatively he capillary tube may be sized such that it has a length that can contact the liquid in the reservoir of a refill at one end and terminate above or adjacent to the at least one wick at the tube's other end without contacting said wick(s).

Preferably the device is provided with a heater that, in use, can transfer heat therefrom to at least a part of said at least one wick. The heater is preferably configured such that in use it may impart enough heat toward the wick to volatilise the liquid held therein and/or thereon but not impart enough heat to boil the liquid.

The refill of liquid that is suitable for use with the device according to the present invention is a refill of liquid containing one or more active materials wherein the refill comprises:

a housing having an inner volume and an outer surface;

at least one reservoir in the inner volume of the housing for holding the liquid;

at least one aperture in the housing sealed by a sealing means;

and at least one wick provided on or connected to the outer surface of the housing, wherein said wick(s) is not extend to the inner volume of the housing.

Unlike all prior art refills which contain a wick in contact either completely or partially with the liquid, in the present invention the wick(s) does not extend to the inner volume of the housing and, thus, cannot be in direct liquid communication with any liquid contained within the reservoir. The at least one wick of the refill of the present invention is not physically able to contact any liquid in the reservoir and can only be used to as a platform to emanate any liquid therefrom once any liquid from the reservoir is transported via a separate transport mechanism from within the inner volume of the housing to the outer surface of the housing where the wick(s) is located. The arrangement of the present invention carries numerous advantages as a consequence. A principal advantage is that no fractionation of the liquid occurs since the wick is being used for emanation purposes only rather than for transport and emanation of the liquid, such as transporting the liquid from the reservoir within the housing and out of the housing to the extremity of the wick which typically protrudes above the exterior of the housing before emanating same to the surrounding environment. The drawback of this transport and emanation is the phenomenon of fractionation, or a variant thereof, which can result when the liquid to be emanated comprises numerous components having different molecular weights and volatilities, the resultant effect can be that the light MW components and/or more volatile components are transported quicker to the end of the wick where emanation typically occurs leaving the heavier MW and/or lower volatility components to be emanated latterly. Such fractionation is particularly noticeable when dealing with a fragranced liquid as the ‘lighter’/‘heady’ notes of the fragrance emanate off quickly leaving the ‘heavier’/‘base’ notes to emanate off later resulting in an experience which is non-uniform and not a true representation of the fragrance the liquid manufacturer intended the user to experience during use. Although potentially less noticeable from a consumer perspective, a further drawback could be when attempting to emanate an insecticide or the like containing one or more active insecticide materials, wherein any fractionation or the like as discussed above can result in non-uniform delivery of the active materials and, potentially, non-uniform levels of protective insecticide in the atmosphere surrounding the refill.

Although a plurality of apertures may be provided, in one preferred arrangement the housing has a single aperture. The aperture is preferably located in an upper wall of the housing. It is to be understood that reference to an “upper wall” is made relative to the other walls of the housing purely for the purpose of spatially describing the refill and, unless otherwise stated, is not to be understood as imparting any restrictive orientation on the refill itself.

The sealing means may be provided by any suitable closure mechanism which permits any liquid contained within the reservoir to be safely and conveniently transported until it is needed. Preferably however, the sealing means is provided by at least one valve. Where a valve(s) is present, said valve(s) is preferably configured to be automatically resealable when not being held open

The at least one valve may be provided by an automatically resealable valve. The valve may be provided in the form of a self-sealing liquid-tight valve, such as a silicone valve, a septum valve or the like. Alternatively the valve may be provided in the form of a movable sealing closure means that is biased towards a closed position by a biasing means, in this arrangement a sealing means such as an O-ring or the like may also be located around the movable sealing closure means to ensure a liquid-tight seal when said closure means is in a closed position and/or the sealing means such as an O-ring may be located in the periphery of the aperture to add a sealing function against any means that are not a part of the refill used to open the closure means.

In one preferred arrangement however at least two valves are provided in the form of a downstream valve (i.e. the valve closest to the reservoir) and an upstream valve. The downstream valve is preferably provided by a self-sealing liquid-tight valve, such as a rubber, rubberised, silicone slit or cross valve or the like, wherein the valve tends toward a closed position when not being held open, or provided by a sealing means biased into a closed position by a deformable biasing means such as a spring means or the like. The upstream valve is preferably provided as an annular ring which is preferably an open ring that is not able to prevent fluid flow therethrough. Preferably the annular ring is supported on a flexible annular skirt. The opening in the annular ring is preferably provided with a diameter of between 1-2000 μm, and more preferably with a diameter of between 50-1500 μm, and even more preferably with a diameter of between 100-1000 μm. The relatively narrow diameters of the preferred ranges permits the annular ring to form a sealing engagement with a capillary tube or the like that enters the ring. Alternatively, the opening in the annular ring is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Said ranges of relatively larger diameters permits the annular ring to form a sealing engagement with an extraction limb that enters the ring; said extraction limb may house one or more liquid conduits and/or one or more capillary tubes or the like. Whilst the annular ring is preferably provided in a circular shape other shapes may be permissible providing the shape is capable of performing the sealing function required of it, such shapes include substantially circular shapes, oval shapes, diamond shapes and such like.

In an alternatively preferred embodiment, the at least one valve is provided in the form of a duckbill valve, wherein the extends into the inner volume of the housing to be operable to be opened when a body, such as a capillary tube or the like, is inserted whilst otherwise be held closed to prevent the loss of any liquid contained therein during transport or storage of the refill. The downstream valve is preferably operable to prevent any fluid within the refill from escaping until the downstream valve is opened and the upstream valve is operable to sealingly engage with liquid extraction means that are part of the emanation device which enter the refill to open a liquid pathway from the refill into the device. In this arrangement the downstream and upstream valves cooperate in that the downstream valve does not need to be optimised to seal against the liquid extraction means that open the valve as the upstream valve can be configured to undertake that task and, vice versa, the upstream valve need not be optimised to form a liquid-tight seal as the downstream seal can be optimised for that task. Furthermore, the open appearance of the upstream valve provides a user with a visual cue to aim either the liquid extraction means from the emanation device or use as a guide when loading the refill onto said means.

In one preferred embodiment the downstream valve and the upstream valve are formed as separate pieces that are held adjacent but spaced apart from each other. In a most preferred embodiment however the downstream valve and the upstream valve are formed as a one piece component.

The refill may be provided with one or more vent holes in the housing, said vent holes being provided in the form of a one-way valve that is in communication with the reservoir but does not facilitate a liquid pathway therefrom rather it solely permits air to enter the reservoir from the outside of the refill to prevent or reduce any build up of negative air pressure within the refill as liquid is removed therefrom. The vent hole(s) may be covered with a gas permeable membrane. Preferably however, the vent hole(s) is not covered and is sized such that the liquid may not escape therefrom or that the rate of escape would be sufficiently low as to not be relevant for the safe operation of the refill with a device configured to operate with the refill to emanate liquid obtained therefrom.

The refill may further comprise one or more diptubes therein. Said diptube(s) may be in communication with the vent hole and/or in communication with the aperture.

Said at least one valve could be adjustable to affect the flow rate of the liquid therethrough. The adjustability may be facilitated manually by a user and/or due to an automated function of a device to which the refill is connected and said automated function may be controlled automatically by the device or may be in response to a user input into the device. Adjustability may be particularly useful when the device is configured to emanate the liquid passively and/or extract the liquid passively such as by gravity feed.

In an alternatively preferred arrangement at least two apertures may be provided in the housing, and even more preferably two apertures are provided. The apertures are preferably located in an upper wall of the housing. It is to be understood that reference to an “upper wall” is made relative to the other walls of the housing purely for the purpose of spatially describing the refill and, unless otherwise stated, is not to be understood as imparting any restrictive orientation on the refill itself.

Each aperture is preferably sealed by the at least one valve wherein each valve is preferably a single automatically resealable valve. Said single valve may be provided in the form of a self-sealing liquid-tight valve, such as a silicone valve, a septum valve or the like. Alternatively said single valve may be provided in the form of a movable sealing closure means that is biased towards a closed position by a biasing means, in this arrangement a sealing means such as an O-ring or the like may also be located around the movable sealing closure means to ensure a liquid-tight seal when said closure means is in a closed position and/or the sealing means such as an O-ring may be located in the periphery of the aperture to add a sealing function against any means that are not a part of the refill used to open the closure means.

In a preferred arrangement however at least one the apertures is sealed by at least two valves provided in the form of a downstream valve (i.e. the valve closest to the reservoir) and an upstream valve. The downstream valve is preferably provided by a self-sealing liquid-tight valve, such as a rubber, rubberised, silicone slit or cross valve or the like, wherein the valve tends toward a closed position when not being held open, or provided by a sealing means biased into a closed position by a deformable biasing means such as a spring means or the like. The upstream valve is preferably provided as an annular ring which is preferably an open ring that is not able to prevent fluid flow therethrough. Preferably the annular ring is supported on a flexible annular skirt. The opening in the annular ring is preferably provided with a diameter of between 1-2000 μm, and more preferably with a diameter of between 50-1500 μm, and even more preferably with a diameter of between 100-1000 μm. The relatively narrow diameters of the preferred ranges permits the annular ring to form a sealing engagement with a capillary tube or the like that enters the ring. Alternatively, the opening in the annular ring is preferably provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm. Said ranges of relatively larger diameters permits the annular ring to form a sealing engagement with an extraction limb that enters the ring; said extraction limb may house one or more liquid conduits and/or one or more capillary tubes or the like. Whilst the annular ring is preferably provided in a circular shape other shapes may be permissible providing the shape is capable of performing the sealing function required of it, such shapes include substantially circular shapes, oval shapes, diamond shapes and such like.

The downstream valve is preferably operable to prevent any fluid within the refill from escaping until the downstream valve is opened and the upstream valve is operable to sealingly engage with liquid extraction means that are part of the emanation device which enter the refill to open a liquid pathway from the refill into the device. In this arrangement the downstream and upstream valves cooperate in that the downstream valve does not need to be optimised to seal against the liquid extraction means that open the valve as the upstream valve can be configured to undertake that task and, vice versa, the upstream valve need not be optimised to form a liquid-tight seal as the downstream seal can be optimised for that task. Furthermore, the open appearance of the upstream valve provides a user with a visual cue to aim either the liquid extraction means from the emanation device or use as a guide when loading the refill onto said means.

In a preferred arrangement however all of the apertures are sealed by at least two valves provided in the form of a downstream valve (i.e. the valve closest to the reservoir) and an upstream valve.

In one preferred embodiment the downstream valve and the upstream valve are formed as separate pieces that are held adjacent but spaced apart from each other. In a most preferred embodiment however the downstream valve and the upstream valve are formed as a one piece component.

The wick(s) is preferably provided on or connected to the outer surface of the upper wall of the housing adjacent the aperture(s). This arrangement is advantageous as it minimises the distance the liquid has to travel from the reservoir to the wick before being emanated, thus improving the response time of the wick when transitioning between emanating and non-emanating functionality.

Preferably however, the wick(s) is located on or connected to the outer surface of the housing such that when the refill is located in a position to extract and emanate liquid contained within the reservoir, then at least one wick is located substantially level with or, most preferably, above the liquid level in the reservoir. Such an arrangement is particularly preferable when extracting liquid from the reservoir via capillary action as an optimum transfer rate of liquid from the reservoir to the wick(s) can be achieved providing the relative height of wick to the height of the liquid level in the reservoir is controlled such that the wick(s) is either substantially level with or slighter higher than the liquid level.

In one embodiment one or more wicks may be provided on or in contact with numerous outer surfaces of the housing such that regardless of the position of the refill when extraction and emanation is to occur at least one wick will be located substantially level with or higher than the liquid level in the reservoir.

Although a plurality of wicks may be provided, preferably a single wick is provided.

The wick may be provided on a collar which is connected to the outer surface of the housing and the collar may be sized to extend away from the outer surface of the housing. However, the wick is preferably sized to remain within contact with the upper wall of the housing without hanging over the edge thereof. Alternatively, the wick may contact both the outer surface of the upper wall of the housing and extend over the edge of the upper wall to also contact one or more side walls of the housing.

The wick(s) may be made of any material that is capable of wicking and emanating a liquid, such suitable materials include plastics materials such as cintered polyethylene, cellulose, woods such as balsa or bamboo, reeds such as rattan.

The housing may be made of any material suitable for retaining a liquid containing one or more active materials, such suitable materials include glass, certain plastics materials and the like. The housing is preferably made from a material that is transparent or at least translucent or is provided with a section thereof adjacent the reservoir(s) that is substantially transparent or translucent to permit a user to easily determine whether the refill contains any liquid and/or the level of said liquid.

According a second aspect of the present invention there is provided therefore a system for emanating a liquid comprising a device and a refill of liquid, wherein the device comprises:

a housing with at least one exit port for emanated liquid therein;

a hollow and elongate extraction limb having at least one capillary tube therein, said at least one capillary tube being open at one end to extract liquid from the refill and open at the other end to deposit said liquid on at least one wick provided only on an outer surface of a refill housing;

and wherein the refill comprises:

a housing having an inner volume and an outer surface;

at least one reservoir in the inner volume of the housing for holding the liquid;

at least one aperture in the housing sealed by a sealing means;

and at least one wick provided on or connected to the outer surface of the housing, wherein said wick(s) is not extend to the inner volume of the housing.

Any of the features described herein may be combined with any of the above aspects in any combination.

### BRIEF DESCRIPTION OF THE DRAWINGS

Embodiments of the invention will now be described, by way of example only, with reference to the following drawings in which:

FIG. 1 shows a perspective view of one embodiment of the refill;

FIG. 2 shows a diagrammatic sectional view of the refill with a capillary tube of a first emanation device engaged therewith;

FIG. 3 shows a diagrammatic sectional view of the refill with a capillary tube of a second emanation device engaged therewith;

FIG. 4 shows a diagrammatic sectional view of the refill with a capillary tube of a third emanation device engaged therewith;

FIG. 5 shows a perspective sectional view of the refill with an extraction limb of a first emanation device engaged therewith;

FIG. 6 shows a perspective sectional view of the refill with an extraction limb of a second emanation device engaged therewith;

FIG. 7 shows a perspective sectional view of the refill with an extraction limb of a third emanation device engaged therewith; and

FIG. 8 shows a sectional view of the refill valve.

### DESCRIPTION OF AN EMBODIMENT

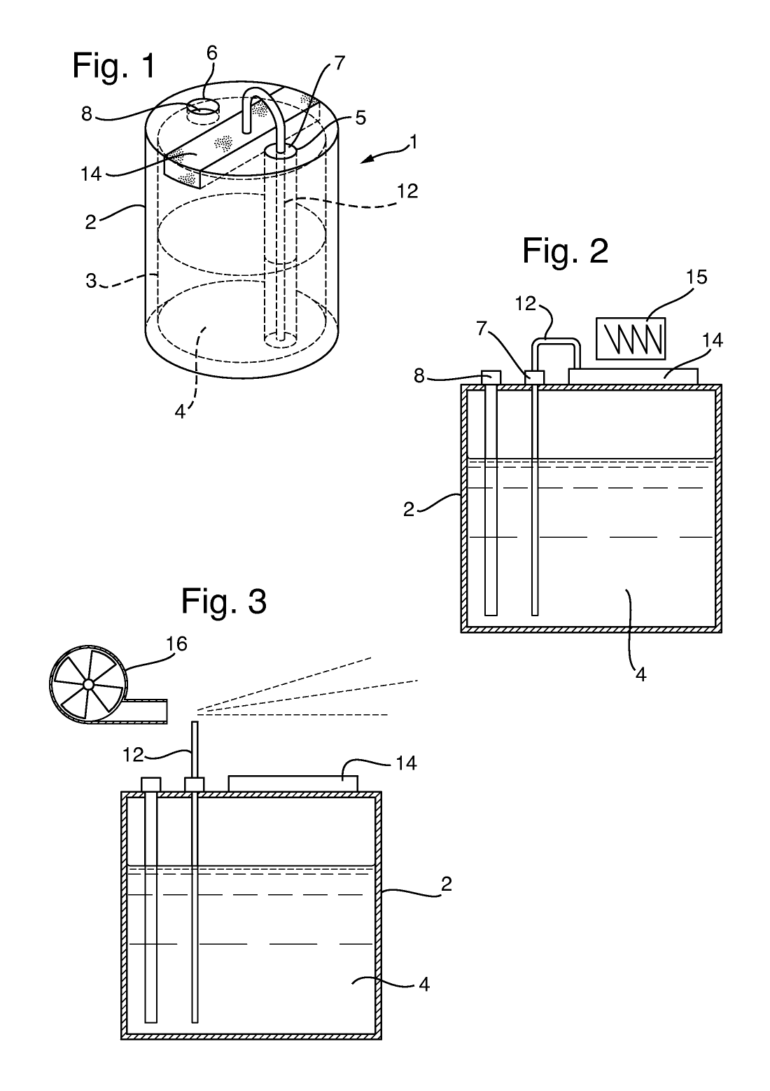

FIG. 1 shows a preferred embodiment of a refill 1 according to the present invention. The refill 1 comprises a housing 2 that surrounds and contains a reservoir 3 of liquid 4 therein. Access to the interior of the refill 1 and the reservoir 3 is provided via one of two apertures 5, 6 which is each sealed by a valve 7,8 (not shown in detail). A capillary tube 12 is sealingly engaged with valve 7. In a preferred arrangement the tube 12 is part of an emanation device that is introduced into the valve 7 and extends toward the base 13 of the reservoir 3. The end of the tube remote from the base 13 is bent through 180° into a U-shape such that it terminates above a wick 14 which is provided entirely on an outer surface of the refill housing 2. The capillary tube 12 is hollow and has an internal diameter of 100 μm +/−50 μm. This diameter permits the liquid 4 to be transported at a desired rate from the reservoir 3 to the wick 14.

FIG. 8 shows the valve 7,8 in greater detail. Each valve 7,8 is an automatically resealable valve system comprising an open annular ring valve 9 suspended by a flexible annular skirt 10 which forms the upstream valve and the valve system further comprises a downstream valve in the form of a rubberised slit valve 11. The downstream slit valve 11 provides a fluid tight closure to prevent the liquid in the reservoir from escaping.

FIG. 2 shows how an emanation device can interact with one or more of the valves 7,8 of the refill 1. The hollow capillary tube 12 is provided operatively connected to the emanation device (the full device is not pictured). The tube 12 is generally elongate and provided at an upper end with a bent U-shape. Preferably the tube is sized to have a diameter that is slightly wider than the diameter of the ring valve 9 of valve 7 such that, in use, when the end of the tube 12 is pushed through the ring valve 9 a liquid-tight connection is made between the tube 12 and the ring valve 9. On travelling further into the valve 7 the end of the tube 12 will meet the slit valve 11 and force it open. Since the tube 12 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In the arrangement shown in FIG. 2 the tube 12 terminates at the end of the U-shape by contacting the wick 14. The wicking action of the wick 14 in combination with the capillary action within the tube 12 will drive the transport of the liquid 4 to the wick 14. Alternatively, the tube 12 could terminate adjacent the wick 14, and preferably just above the wick such that a drop will form at the end of the tube 12 and will grow in size until it reaches a critical mass where it can no longer adhere to the tube and falls on to the wick 14. Although not shown in FIG. 2, the device may also open valve 8 in order to permit air to be returned to the reservoir 3 and thus prevent any retarding of the transport of liquid out of the refill 1 due to build up of negative pressure as liquid 4 leaves the reservoir 3.

A heater 15 in the device is located adjacent the wick 14 to, in use, direct heat toward the wick and accelerate the volatilisation of the liquid therefrom and out of an exit port (not shown) in the device and into the surrounding environment.

FIG. 3 shows an emanation device (the full device is not pictured) that uses an air pump 16 to aid the extraction of the liquid 4 from the reservoir 3. A hollow capillary tube 12 is provided operatively connected to the emanation device. The tube 12 is generally elongate with an upper end that terminates adjacent the air pump 16. Preferably the tube is sized to have a diameter that is slightly wider than the diameter of the ring valve 9 of valve 7 such that, in use, when the end of the tube 12 is pushed through the ring valve 9 a liquid-tight connection is made between the tube 12 and the ring valve 9. On travelling further into the valve 7 the end of the tube 12 will meet the slit valve 11 and force it open. Since the tube 12 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In addition to the transporting of liquid via capillary action, the air pump 16 is configured to blow air across the top of the tube 12 to cause liquid to travel up the conduit by the Venturi effect as well. The flow of air is additionally instrumental in forcing the liquid against a mechanical break up means (not shown) and out of an exit port in the device into the surrounding environment. Although not shown in FIG. 3, the device may also open valve 8 in order to permit air to be returned to the reservoir 3 and thus prevent any retarding of the transport of liquid out of the refill 1 due to build up of negative pressure as liquid 4 leaves the reservoir 3.

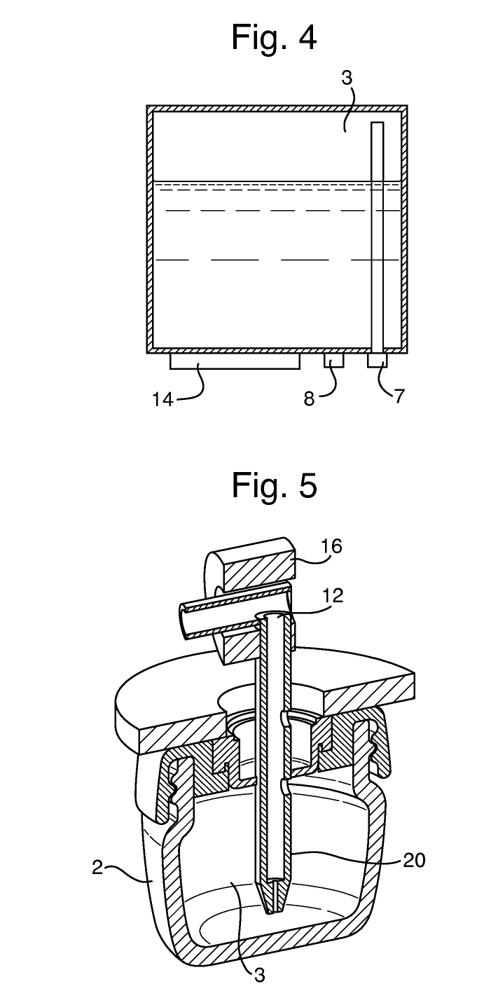

A gravity-feed emanation device (full device not pictured) is shown in FIG. 4. In this arrangement the refill 1 is loaded into the device in an inverted orientation. The tube 12 is generally elongate with an upper end that terminates adjacent toward an inner surface of the reservoir remote from the valves 7,8. Preferably the tube 12 is sized to have a diameter that is slightly wider than the diameter of the ring valve 9 of valve 7 such that, in use, when the end of the tube 12 is pushed through the ring valve 9 a liquid-tight connection is made between the tube 12 and the ring valve 9. On travelling further into the valve 7 the end of the tube 12 will meet the slit valve 11 and force it open. Since the tube 12 and the ring valve 9 have formed a liquid-tight connection any liquid that is able to flow past the slit valve 11 will be prevented from leaking out of the refill 1. To extract the liquid 4 from the refill the device has an additional extraction means (not shown) that opens valve 8 to permit the liquid to flow in a downward direction powered by gravity and into the device. The tube 12 also air to flow into the reservoir in the head-space formed by the dropping liquid level to prevent any retarding of the transport of liquid out of the refill 1 due to build up of negative pressure as liquid 4 leaves the reservoir 3.

Whereas FIGS. 2-4 show a refill 1 having two valves 7,8, FIGS. 5-7 shown a refill with a single valve 7 and how emanation devices using differing extraction and emanation methods can interact with same.

In FIG. 5 an emanation device (the full device is not pictured) that uses an air pump 16 to aid the extraction of the liquid 4 from the reservoir 3 is shown. The device is provided with a hollow and elongate extraction limb 20 that surrounds a hollow capillary tube 12. The tube 12 is also elongate with an upper end that terminates adjacent the air pump 16. Preferably the limb 20 is sized to have a diameter that is slightly wider than the diameter of the ring valve 9 such that, in use, when the end of the limb 20 is pushed through the ring valve 9 a liquid-tight connection is made between the limb 20 and the ring valve 9. On travelling further into the valve 7 the end of the limb 20 will meet the slit valve 11 and force it open. Since the limb 20 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In addition to the transporting of liquid via capillary action, the air pump 16 is configured to blow air across the top of the tube 12 to cause liquid to travel up the conduit by the Venturi effect as well. The flow of air is additionally instrumental in forcing the liquid against a mechanical break up means (not shown) and out of an exit port in the device into the surrounding environment. Although not shown in FIG. 5, the refill 1 may be provided with a vent hole to permit air to return to the refill to prevent any retarding of the transport of liquid out of the refill 1 due to build up of negative pressure as liquid 4 leaves the reservoir 3.

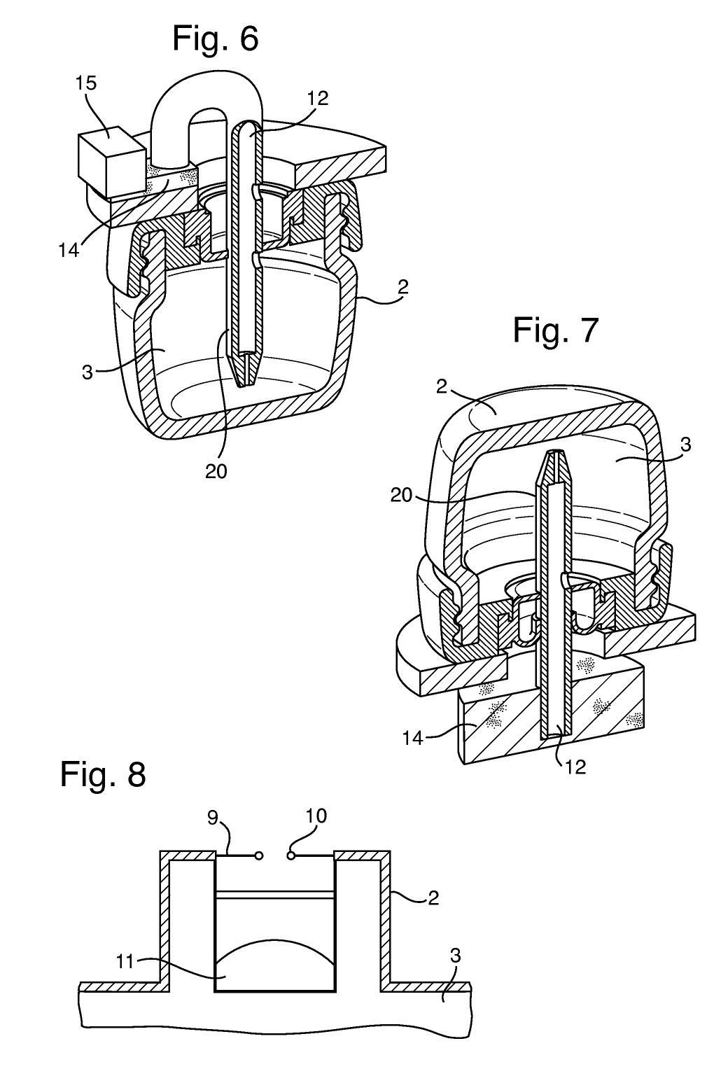

FIG. 6 shows a device that uses a combination of capillary action and a heater to extract and emanate liquid from the refill. The device (the full device is not pictured) is provided with a hollow extraction limb 20 that surrounds the hollow capillary tube 12. The extraction limb 20 is generally elongate and the tube 12 is also generally elongate but provided at an upper end with a bent U-shape. Preferably the limb 20 is sized to have a diameter that is slightly wider than the diameter of the ring valve 9 such that, in use, when the end of the limb 20 is pushed through the ring valve 9 a liquid-tight connection is made between the limb 20 and the ring valve 9. On travelling further into the valve 7 the end of the limb 20 will meet the slit valve 11 and force it open. Since the limb 20 and the ring valve 9 have formed a liquid-tight connection any liquid that is able to flow past the slit valve 11 will be prevented from leaking out of the refill 1. The tube 12 will on contact with the liquid 4 immediately begin to transport the liquid up the tube 12 using capillary action. In the arrangement shown in FIG. 6 the tube 12 terminates at the end of the U-shape by contacting the wick 14. The wicking action of the wick 14 in combination with the capillary action within the tube 12 will drive the transport of the liquid 4 to the wick 14. Alternatively, the tube 12 could terminate adjacent the wick 14, and preferably just above the wick such that a drop will form at the end of the tube 12 and will grow in size until it reaches a critical mass where it can no longer adhere to the tube and falls on to the wick 14.

A heater 15 in the device is located adjacent the wick 14 to, in use, direct heat toward the wick and accelerate the volatilisation of the liquid therefrom and out of an exit port (not shown) in the device and into the surrounding environment.

Although not shown in FIG. 6, the refill 1 may be provided with a vent hole to permit air to return to the refill to prevent any retarding of the transport of liquid out of the refill 1 due to build up of negative pressure as liquid 4 leaves the reservoir 3.

A gravity-feed emanation device (full device not pictured) is shown in FIG. 7. In this arrangement the refill 1 is loaded into the device in an inverted orientation. The device is provided with a hollow and elongate extraction limb 20 containing at least one perforation (not shown) therein to permit access to the hollow interior of the limb 20. The extraction limb 20 is provided with a hollow capillary tube 12 therein. Preferably the limb 20 is sized to have a diameter that is slightly wider than the diameter of the ring valve 9 of valve 7 such that, in use, when the end of the limb 20 is pushed through the ring valve 9 a liquid-tight connection is made between the tube 12 and the ring valve 9. On travelling further into the valve 7 the end of the limb 20 will meet the slit valve 11 and force it open. Since the limb 20 and the ring valve 9 have formed a liquid-tight connection any liquid that flows past the slit valve 11 will be prevented from leaking out of the refill 1. Once the limb 20 is inside the refill as shown in FIG. 7, liquid 4 is able to flow through the perforation(s) into the hollow interior of the limb 20 and flow in a downward direction powered by gravity and into the device. The tube 12 permits air to flow into the reservoir in the head-space formed by the dropping liquid level to prevent any retarding of the transport of liquid out of the refill 1 due to build up of negative pressure as liquid 4 leaves the reservoir 3.

Although not shown, the end of the housing 1 containing the valve 7 or valves 7,8 may be substantially square in shape and guide means (not shown) in a chassis (not shown) of each emanation device) could cooperate with grooves in the refill housing (not shown) to ensure that the refill could only be engaged with the chassis if the limb 11 is correctly aligned to open the valve 7 or valves 7,8. Although not illustrated, the end of the refill housing containing the valve 7 or valves 7,8 could be shaped such that the loading thereof into the chassis was only possible via a single orientation of that end of the refill.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A system for emanating a liquid comprising a device and a refill of liquid, wherein the device comprises:
a housing with at least one exit port for emanated liquid therein;
a hollow and elongate extraction limb having at least one capillary tube therein, said at least one capillary tube being open at one end to extract liquid from the refill and open at the other end to deposit said liquid on at least one wick provided only on an outer surface of a refill housing; and wherein the refill comprises:
a housing having an inner volume and an outer surface;
at least one reservoir in the inner volume of the housing for holding the liquid;
at least one aperture in the housing sealed by a sealing means;
and at least one wick provided on or connected to the outer surface of the housing, wherein said wick(s) does not extend to the inner volume of the housing, and wherein the refill has no liquid transport mechanism to transport the liquid from the reservoir to said at least one wick.

2. A system according to claim 1, wherein the extraction limb has a single capillary tube therein.

3. A system according to claim 1, wherein the extraction limb is provided with one or more vent holes.

4. A system according to claim 1, wherein the extraction is the capillary tube.

5. A system according to claim 1, wherein the extraction limb is provided with a diameter of between 0.1-20.0 mm, and more preferably with a diameter of between 3.0-15.0 mm, and even more preferably with a diameter of between 4.0-10.0 mm, and most preferably with a diameter of between 5.0-7.0 mm.

6. A system according to claim 1, wherein the capillary tube(s) within the extraction limb is provided with a diameter of between 1-2000 μm, and more preferably with a diameter of between 50-1500 μm, and even more preferably with a diameter of between 100-1000 μm, and most preferably with a diameter of 100 μm+/−50 μm.

7. A system according to claim 1, wherein the extraction limb has one end in contact with the device and the end remote therefrom is tapered.

8. A system according to claim 1, wherein the capillary tube(s) is bent at its end adjacent to the device through an angle of between 45°-180°.

9. A system according to claim 1, wherein the capillary tube is bent through 180° to be located, in use, directly over a wick on the outer surface of the refill.

10. A system according to claim 1, wherein the device is provided with a heater that, in use, can transfer heat therefrom to at least a part of said at least one wick.

11. A system according to claim 10, wherein the heater is configured such that in use it imparts enough heat toward the wick to volatilise the liquid held therein and/or thereon but not impart enough heat to boil the liquid.
